(19) United States
(12) Patent Application Publication
(10) Pub. No.: US 2003/0180893 A1
(43) Pub. Date: Sep. 25, 2003

(54) Biotransformation of biologically active compounds made of various classes of chemical substance by means of laccase and manganese peroxidase enzymes

(76) Inventors: Frieder Schauer, Greifswald (DE); Ulrike Lindequist, Greifswald (DE); Annett Schafer, Greifswald (DE); Julich Wolf-Dieter, Greifswald (DE); Elke Hammer, Wackerow (DE)

Correspondence Address: JACOBSON HOLMAN PLLC, 400 SEVENTH STREET N.W., SUITE 600, WASHINGTON, DC, 20004, US

(21) Appl. No.: 10/311,977

(22) PCT Filed: Jun. 23, 2001
(86) PCT No.: PCT/EP01/07152

(30) Foreign Application Priority Data
Jun. 23, 2000 (DE) 10029671.8

(51) Int. Cl. C12P 19/00; C12P 1/00

(52) U.S. Cl. 435/72; 435/41

(57) ABSTRACT

A process for the preparation of biologically active compounds, wherein active substances having additional functional groups and a modified spectrum of activity and modified application properties are obtainable from medicinal substances and plant protective agents as substrates which bear at least one amino or hydroxy functional group by using a one-electron reaction catalyzed by enzymes or compositions having enzymatic activity and a broad spectrum of substrates, characterized in that free-radical forming enzymes are employed as enzymes and/or supernatants of ligninolytic fungi in solution are employed as compositions having enzymatic activity, wherein the following can be introduced as additional functional groups:

A) polyfunctional synthones which provide the active substance with an altered dissolving behavior in aqueous and lipophilic systems and a high interfacial activity;

B) aromatic molecules, heteroaromatic compounds;

C) heterocyclic compounds;

D) active substances having an independent biological activity;

E) active substances combining an independent activity and surfactant properties in one molecule;

which yield coupling products being covalently linked with the starting materials and having an altered partition behavior which enable polyfunctional interactions with the target organism.

The invention relates to novel biologically active compounds, processes for the preparation thereof, and their use. From antibiotics and chemotherapeutics, coupling products which have either an improved antimicrobial activity or improved application properties can be obtained in a biotransformation catalyzed by the enzymes laccase or manganese peroxidase.

### PRIOR ART

With respect to structure and biological activity, antibiotics form a heterologous group of compounds which are among those therapeutics mostly employed in human and veterinary medicine and in plant protection. Antibiotics include substances formed by microorganisms and derivatives formed therefrom which, when applied intrasomatically, can control infectious diseases. In addition to antibacterial antibiotics, antibiotics having antifungal, antiviral, trypanocidal or cancerostatic properties have been discovered. Due to the application of antibiotics prepared on a fermentative route in cancer chemotherapy, their original definition has been extended, which has also been done due to their application in animal breeding as ergotropics and in plant protection as herbicides, insecticides, pesticides, acaricides, nematocides, molluscicides (U. Grafe: Biochemie der Antibiotika, Spektrum Akademischer Verlag, Heidelberg, Berlin, N.Y., 1992). The search for new cellular target sites for antibiotics and similar biologically active substances has not been completed yet and leads to new possible applications in human and veterinary medicine and, for example, in plant protection.

Finally, the extensive modification of naturally occurring antibiotics by partial synthesis and the total chemical synthesis thereof led to medicinal substances which considerably exceed the original potential of natural substances. Finally, there were also found products of total synthesis without any relation with microbial products, but which are equivalent with the classical antibiotics with respect to their effects on microorganisms and their mechanism of action (U. Grafe: Biochemie der Antibiotika, Spektrum Akademischer Verlag, Heidelberg, Berlin, N.Y., 1992).

Hereinafter, the term “antibiotics” is used in this extensive sense for biologically active substances having selective effects at different sites of action of pathogenic microorganisms (bacteria, fungi, viruses) and phylogenetically superior organisms (e.g., rickettsiae, trypanosomes, tumor cells or parasites).

Antibiotics having various biological activities for different fields of application are constantly modified by chemical reactions in order to adapt them to new tasks. No other group of antibiotics has met with such an extensive semisynthetic modification as that of β-lactam antibiotics. The discovery of 6-aminopenicillanic acid as a parent substance of penicillins and of 7-aminocephalosporanic acid as a parent substance of cephalosporins led in a new phase of modification directed to derivatives with optimized effectiveness which has not been completed yet.

The advantages of the originally discovered penicillins and cephalosporins, such as very low toxicity and high bactericidal effect, were adapted to the increasing demands by continuous chemical modification. U.S. Pat. No. 5,695,951 and U.S. Pat. No. 5,939,299 disclose methods for chlorinating the cephalosporin derivative cephalexin using a haloperoxidase. A method for the enzymatic introduction of halogens using haloperoxidases is described in WO-A-00/15771.

However, these chemical and biochemical modifications of β-lactam antibiotics could not keep pace with the development of resistance in the pathogenic microorganisms, so that numerous germs, especially multiresistant staphylococci, can no longer by treated with the currently available β-lactam antibiotics.

Laccases (E.C. 1.10.3.2) are enzymes which catalyze the oxidation of a substrate. They are obtained from microbes, plants and animals. In nature, laccases and peroxidases play an important role, for example, in the biological degradation of lignin, which is why they can be isolated, inter alia, from white rot fungi. Such ligninolytic enzymes are capable of oxidatively converting various environmentally hazardous substances (Jonas, U., Hammer, E., Schauer F., Bollag, J.-M.: Biodegradation 1998; 8: 321-328; Bollag, J.-M., Shuttleworth, K. L., Anderson, D. H.: Appl. Environ. Microbiol. 1988; 54: 3086-3091; Bumpus, J. A., Aust, S. D.: Bioessays 1986; 6: 166-170; Bumpus, J. A., Tien, M., Wright, D. A.: Science 1985; 228: 143-147).

DE-A-197 26 241 discloses an extended multicomponent system for waste-water treatment which may also contain, inter alia, oxidoreductases obtained from white rot fungi. Inter alia, laccase may also be employed for waste-water treatment in a concentration of from 0.001 to 1 U/ml. The multicomponent system developed for waste-water treatment may also be employed for organic syntheses, for example, for the oxidation of unsaturated aliphatics or for the oxidation of alcohols to aldehydes. According to DE-A-24 02 452, it has been found advantageous to effect laccase-mediated oxidations in a two-phase system of water and an organic solvent.

U.S. Pat. No. 5,389,356 and U.S. Pat. No. 5,468,628 describe methods of selective oxidation or reduction in which a peroxidase serves as a catalyst generating free radicals. This method is suitable, for example, for degrading carbon tetrachloride.

It is further known to oxidize phenols using laccase or peroxidase, wherein the reaction solutions are antimicrobially effective and can be employed in industrial processes for germ control. A specific application of mild oxidation using tyrosinases and laccases is described in U.S. Pat. No. 6,015,683. Thus, using laccase, a colorant reagent is prepared as an intermediate which enables the spectrophotometric detection of acetaminophen in aqueous solutions.

The known antimicrobial effect of laccases in the presence of oxygen is also due to their oxidative power. Also based on oxidation is the formation of antimicrobially effective phenoxazinone derivatives under the influence of laccase. Laccase and manganese peroxidase are already being used economically for a wide variety of purposes. They bleach lignin-containing material and are therefore employed in the paper industry. From WO-A-95/01426, it is known that organic-chemical compounds consisting of at least two aromatic rings can enhance the activity of laccase. This enhancement was utilized for bleaching dyes in solutions or lignin and lignin-containing materials.

U.S. Pat. No. 4,478,683 describes the prevention of the growth of microorganisms in industrial waste-water treatment by the addition of laccases and peroxidases.

In a few cases, laccase and manganese peroxidase have been employed for effecting a coupling between different biological compounds. Since a preferential attack on functional groups essential to activity is to be anticipated, a deterioration of effectiveness is to be expected as a rule. This is probably the reason why experiments for the use of manganese peroxidase and laccase for the derivatization of antibiotics are limited to a few examples. In the examples realized to date, the sought object of enhancement of the antimicrobial effect was not achieved. For example, laccase was employed for linking antibiotics of the aureolic acid group with hydroquinones (Anyanwutaku, I. O., Petrowski, R. J., Rosazza, I. P.: Bioorg. Med. Chem. 1994; 2: 543-551). This involved the loss the of biological activity of the starting materials. Agematu et al. (Biosci. Biotechnol. Biochem. 57 (8), 1387-1388 (1993)) describe the transformation of 7-(4-hydroxyphenylacetamido)cephalosporinic acid by a laccase-catalyzed phenolic oxidation. In this case too, the reaction product was less effective than the original cephalosporin. Uemtsu et al. (Jpn. Kokai Tokyo JP 05,163,281) employ oxidases for the oxidation of cephalosporins.

From penicillin methyl ester, three different types of dimers which are all biologically inactive are obtained under the influence of laccase and under certain conditions (Agematu, H., Tsuchida, T., Kominato, K., Shibamoto, N., Yoshioka, T., Nishida, H., Okamoto, R., Shin, T., Murano, S.: J. Antibiot. Tokyo 1993; 46: 141-148). The enzymatic synthesis of dimers of phenol and of penicillin is summarized by Agemato (Baiosainsu to Indasutori 1996, 54, 715-718).

Enzymes which have been employed to date for the modification of antibiotics, such as haloperoxidase, have a limited spectrum of substrates. The chlorination mediated by haloperoxidases can be employed only in specific cases.

When microorganisms are subjected to the permanent action of sublethal antibiotic concentrations, they develop antibiotic resistances as a part of their genetically programmed adaptation mechanism to changing environmental conditions, i.e., they are no longer inhibited by the original minimum inhibition concentration of the active substance in question. Since the action of sublethal doses can never be completely avoided, a resistance will be developed by pathogenic microorganisms against each new antibiotically active substance. Active substances formed under the influence of peroxidases or laccases and having effectiveness against germs having become resistant have not yet been described.

Due to the increasingly important resistance problems, there is still a high demand for antibiotics, which is not covered by the active substances corresponding to the prior art.

This is true, in particular, for the use of antibiotics in infectious diseases which are difficult to control clinically. In infections with Gram-positive germs, only glycopeptide antibiotics are still sufficiently active currently. Gram-positive pathogens, such as staphylococci and enterococci, increasingly develop multiple resistances. But also infectious diseases caused by Gram-negative pathogens will escape safe clinical control in the future. Also in veterinary medicine, infectious diseases of farm animals with multiresistant staphylococci are becoming increasingly more threatening. An additional danger looms from the possible transfer to humans of germs having become resistant.

The object of the invention is to further develop biologically active substances using biotransformation and to make them useful for medicinal use as coupling products with other antimicrobially active substances. In particular, it is the object of the invention to cover the demand for measures which exists due to the increasing development of resistances by bacteria towards conventional antibiotics, especially in the therapy of infectious diseases, in human and veterinary medicine, by developing a biotransformation process by which as yet unknown active substances can be obtained from known antimicrobially active substances.

According to the invention, this object is achieved by a process for the preparation of biologically active compounds, wherein active substances having additional functional groups and a modified spectrum of activity and modified application properties are obtainable from medicinal substances and plant protective agents as substrates which bear at least one amino or hydroxy functional group by using a one-electron reaction catalyzed by enzymes or compositions having enzymatic activity and a broad spectrum of substrates, characterized in that free-radical forming enzymes are employed as enzymes and/or supernatants of ligninolytic fungi in solution are employed as compositions having enzymatic activity, wherein the following can be introduced as additional functional groups:

A) polyfunctional synthones which provide the active substance with an altered dissolving behavior in aqueous and lipophilic systems and a high interfacial activity;

B) aromatic molecules, heteroaromatic compounds;

C) heterocyclic compounds;

D) active substances having an independent biological activity;

E) active substances combining an independent activity and surfactant properties in one molecule;

which yield coupling products being covalently linked with the starting materials and having an altered partition behavior which enable polyfunctional interactions with the target organism.

Preferably, the substrates are β-lactam antibiotics, tetracyclin antibiotics, anthracyclin antibiotics, polyene antibiotics, aminoglycosides, benzofuran derivatives, sulfonamides, quinoid compounds and organic acids.

According to the invention, converting these antimicrobially active substances by a process according to claims 1 to 12 can yield compounds which are characterized in that an activity which could not be detected in the starting materials, preferably against multiresistant germs, is achieved. The compounds which can be obtained from the conversion by the process according to the invention may further be characterized in that the development of resistances is rendered more difficult by the covalent linking of molecules having different mechanisms of action and different molecular target sites. However, it is also according to the invention to achieve an improvement of the application properties by introducing additional hydrophilic and/or hydrophobic components without altering the spectrum of activities.

According to the invention, those enzymes are preferably employed which belong to the following classifications according to the International Enzyme Nomenclature (Enzyme Nomenclature, Academic Press, Inc., 1992, p. 24-154): EC 1.10.3.2 (laccase), EC 1.11.1.13 (manganese peroxidases), EC 1.11.17 (peroxidases), EC 1.14.99.1 (monophenol monooxygenase), EC 1.10.3.3 (ascorbate oxidase).

If the process according to the invention is performed in an aqueous solution, the latter preferably has a pH value of from 2 to 8, preferably from 3 to 5, and temperatures of between 5° C. and 60° C.

As the polyfunctional synthones mentioned under A), there may be used:

C₁-C₁₈ Alkyl, C₁-C₁₈ alkenyl, C₁-C₁₈ alkynyl, C₁-C₁₈ alkoxy, C₁-C₁₈ oxycarbonyl, C₁-C₁₈ oxoalkyl, C₁-C₁₈ alkylsulfanyl, C₁-C₁₈ alkylsulfonyl, C₁-C₁₈ alkylimino or alkylamino synthones.

According to the invention, the aromatic molecules mentioned under B) are preferably the following: mono-, di- or polycyclic aromatic synthones, each optionally provided with one or more functional groups or substituents selected from the group of halogens; sulfo; sulfone; sulfamino; sulfanyl; amino; amido; nitro; azo; imino; carboxy; cyano; formyl; hydroxy; halocarbonyl; carbamyl; carbamidoyl; phosphone; phosphonyl; C₁₋₁₈ alkyl; C₁₋₁₈ alkenyl; C₁₋₁₈ alkinyl; C₁₋₁₈ alkoxy; C₁₋₁₈ oxycarbonyl; C₁₋₁₈ oxoalkyl; C₁₋₁₈ alkylsulfanyl; C₁₋₁₈ alkylsulfonyl; C₁₋₁₈ alkylimino or alkylamino substituents. Especially preferred mono-, di- or polycyclic aromatic compounds are those selected from the group consisting of anthracene, azulene, benzene, benzofuran, benzothiazole, benzothiazoline, carboline, carbazole, cinnoline, chroman, chromene, chrysene, fulvene, furan, imidazole, indazole, indene, indole, indoline, indolizine, isothiazole, isoquinoline, isoxazole, naphthalene, naphthylene, naphthylpyridine, oxazole, perylene, phenanthrene, phenazine, phthalizine, pteridine, purine, pyran, pyrazole, pyrene, pyridazine, pyridazone, pyridine, pyrimidine, pyrrole, quinazoline, quinoline, quinoxaline, sulfonyl, thiophene and triazine, each of which may be substituted. Examples of such compounds, without excluding others, are aromatic diamines, aminophenols, phenols, naphthoquinones and 2,5-dihydroxylated benzoic acid derivatives. Further, without excluding others, the following compounds may be used:

3,4-Diethoxyaniline, 2-methoxy-p-phenylenediamine, 1-amino-4-beta-methoxyethylaminobenzene (N-beta-methoxyethyl-p-phenylenediamine), 1-amino-4-bis-(beta-hydroxy-ethyl)aminobenzene, 2-methyl-1,3-diaminobenzene (2,6-diaminotoluene), 2,4-diaminotoluene, 1-amino-4-sulfonatobenzene, 1-N-methylsulfonato-4-aminobenzene, 1-methyl-2-hydroxy-4-aminobenzene (3-amino-o-cresol), 1-methoxy-2,4-diaminobenzene (2,4-diaminoanisole), 1-ethoxy-2,3-di-aminobenzene (2,4-diaminophenetole), 1-beta-hydroxyethyloxy-2,4-diaminobenzene (2,4-diaminophenoxyethanol), 1,3-dihydroxy-2-methylbenzene (2-methylresorcinol), 1,2,4-trihydroxybenzene, 1,2,4-trihydroxy-5-methylbenzene (2,4,5-trihydroxytoluene), 2,3,5-trihydroxytoluene, 4,8-disulfonato-1-naphthol, 3-sulfonato-6-amino-1-naphthol, 1,4-phenylenediamine, 2,5-diaminotoluene, 2-chloro-1,4-phenylenediamine, 2-aminophenol, 3-aminophenol, 4-aminophenol, 1,3-phenylenediamine, 1-naphthol, 2-naphthol, 4-chlororesorcinol, 1,2,3-benzenetriol (pyrogallol), 1,3-benzenediol (resorcinol), 1,2-benzenediol (pyro-catechol), 2-hydroxycinnamic acid, 3-hydroxycinnamic acid, 4-hydroxycinnamic acid, 2,3-diaminobenzoic acid, 2,4-diaminobenzoic acid, 3,4-diaminobenzoic acid, 3,5-diaminobenzoic acid, methyl 2,3-diaminobenzoate, ethyl 2,3-diaminobenzoate, isopropyl 2,3-diaminobenzoate, methyl 2,4-diaminobenzoate, ethyl 2,4-diaminobenzoate, isopropyl 2,4-diaminobenzoate, methyl 3,4-diaminobenzoate, ethyl 3,4-diaminobenzoate, isopropyl 3,4-diaminobenzoate, methyl 3,5-diaminobenzoate, ethyl 3,5-diaminobenzoate, isopropyl 3,5-diaminobenzoate, N,N-diethyl-3,4-diaminobenzoic amide, N,N-dipropyl-3,4-diaminobenzoic amide, 3,4-dihydroxybenzaldehyde, anthranilic acid, 4-aminobenzoic acid, ethylhydroquinone, mandelic acid, o-nitrobenzaldehyde, 1,5-dihydroxynaphthalene, 2,6-dihydroxynaphthalene, 2,3-dihydroxynaphthalene, benzylimidazole, salicylic acid, 3-aminosalicylic acid, 4-aminosalicylic acid, 5-aminosalicylic acid, methyl 3-aminosalicylate, methyl 4-aminosalicylate, methyl 5-aminosalicylate, ethyl 3-aminosalicylate, ethyl 4-aminosalicylate, ethyl 5-aminosalicylate, propyl 3-aminosalicylate, propyl 4-aminosalicylate, propyl 5-aminosalicylate, salicylic amide, 4-aminothiophenol, 4-hydroxythiophenol, N,N-dimethyl-1,4-phenylenediamine, N,N-diethyl-1,4-phenylenediamine, N-phenyl-1,2-phenylenediamine, 6-amino-2-naphthol, 3-amino-2-naphthol, 5-amino-1-naphthol, 1,2-phenylenediamine, 4-aminoquinaldine, 2-nitroaniline, 3-nitroaniline, 2-chloroaniline, 3-chloroaniline, 4-chloroaniline, 4-aminoacetanilide, alizarin, 1-anthramine (1-aminoanthracene), 1-aminoanthraquinone, anthraquinone, 2,6-dihydroxyanthraquinone, 1,5-dihydroxyanthraquinone (anthrarufin).

According to the invention, as the heterocyclic compounds mentioned under C), there are preferably employed pyrrolidine, pyridine, quinolizidine, quinoline, isoquinoline, indole, acridine, quinazoline and purine, each optionally provided with one or more functional groups or substituents selected from the group of halogens, sulfo, sulfone, sulfamino, sulfanyl, amino, amido.

According to the invention, as the active substances according to D), there may be used, in particular, imidazoles, azoles, flavones, isoflavones, tetracyclines, amino acid analogues and nucleotide analogues.

In the process according to the invention, a change of optimum pH can be achieved by the use of recombinant laccases.

According to the invention, the coupling reaction can be optionally performed in the presence of at least one mediator selected from the group of hydroxylamines and/or hydroxamic acids and optionally a mediator selected from the group of amides.

Optionally, the reaction products may be stabilized after completion of the reaction.

The present invention also relates to compounds obtainable from active substances from different classes of substances by a biotransformation according to the invention.

The biologically active compounds according to the invention can be employed in human and veterinary medicine and in plant protection as a sole active ingredient or in the form of combination preparations.

The biologically active compounds according to the invention can be employed, in particular, for controlling infectious diseases, preferably as an agent having antimicrobial and antiviral effects for topical and/or systemic application, in infectious diseases with multiresistant Gram-positive or Gram-negative germs, as an antimycotic agent for topical and/or systemic application, and as a cytostatic agent in human and veterinary medicine and in plant protection as herbicides, insecticides or molluscicides.

The invention also relates to medicaments containing one or more of the compounds according to the invention.

The invention also relates to the use of one or more of the compounds according to the invention for the preparation of a medicament for treating infectious diseases with Gram-positive pathogens.

The process according to the invention also results in novel plant protective agents which contain at least one of the compounds according to the invention.

The process according to the invention results in biologically active compounds which can be employed as cytostatic agents.

The compounds obtained upon biotransformation can be employed alone or in combination with one another.

According to the invention, by introducing additional hydrophilic and/or hydrophobic components, an improvement of the application properties of the starting compounds can be achieved. The compounds according to the invention may be employed, in particular, as surface-active substances.

The compounds according to the invention obtainable from antimicrobially active agents by a conversion by the process according to the invention advantageously exhibit an activity which cannot be detected in the starting substances, preferably against multiresistant germs.

The compounds according to the invention which can be obtained from antimicrobially active substances by a conversion by the process according to the invention render the formation of resistances more difficult by the covalent linking of molecules having different mechanisms of action and different molecular target sites, which is achieved by the action of free-radical forming enzymes.

Based on a coupling reaction catalyzed by laccases and by free-radical forming enzymes similar to laccase, according to the invention, a generally applicable process of biotransformation becomes available with which various biologically active substances containing amino or hydroxy functional groups can be interlinked to achieve an improvement of biological activity and/or an improvement of application properties. Essential characterizing steps in the preparation of the novel compounds are enzymatically catalyzed one-electron oxidations, an enzymatically catalyzed formation of superoxide free radical, or an enzymatically catalyzed formation of aryl free radical.

Surprisingly, it has been found that these enzymatically catalyzed reactions enable active substances to be obtained having a biological activity which is clearly superior to that of the starting materials. For these enzymatically catalyzed reaction steps, according to the invention, the presence of, in particular, laccase or enzymes similar to laccase is required. In this connection, “laccases and enzymes similar to laccase” means, in particular, the enzymes summarized under the classification of EC 1.10.3.2 as well as catechol oxidase (EC 1.10.3.1), bilirubin oxidase (EC 1.3.3.5), monophenol monooxygenase (EC 1.14.18.1), and o-aminophenol oxidase (1.10.3.4). The laccases are preferably obtained from fungi from the genera Pycnoporus, Trametes, Coriolus, Collybia, Fomes, Lentinus, Pleurotus, Rhizoctonia, Aspergillus, Neurospora, Podospora, Phlebia or Myceliophthora, or on a biotechnological route. It is possible to employ other ligninolytical enzymes instead of laccase, such as peroxidases, especially manganese peroxidase (1.11.1.13) and lignin peroxidase (1.11.1.14).

By the process according to the invention, active substances from different classes of chemical substances can be connected with each other. Advantageously, the active substances to be connected have biological activities with different target sites. However, it is also possible to connect a biologically active substance with a substance having no independent antimicrobial activity using the process according to the invention in order to improve the application properties, for example, the partition behavior. By the action of laccase or manganese peroxidase on antimicrobially active starting materials according to the invention, novel biologically active compounds are obtained which have improved application properties. It has been completely surprising that, in particular, p-lactam antibiotics, such as penicillins, cephalosporins and carbapenems, should be convertible by the process according to the invention with retention of their antimicrobial activity. Sulfonamide antibiotics as well as variously substituted salicylic acids could also be transformed with the process according to the invention, and the products showed antimicrobial properties. It is particularly advantageous to connect β-lactam and sulfonamide antibiotics as well as substituted salicylic acids with quinoid and hydroquinoid active substances by a laccase-catalyzed reaction. The novel active substances obtainable by the biotransformation according to the invention exhibit antimicrobial activity even against multiresistant germs. Thus, the requirements for applying the novel active substances in human and veterinary medicine for controlling infectious diseases are met. The novel active substances obtainable by the process according to the invention can be employed both topically and systemically, alone or in the form of combination preparations.

In the following, the invention will be illustrated in more detail by some Examples without being limited of such Examples.

### EXAMPLE 1

Preparation of Novel Antimicrobially Active Substances by Coupling 2,5-dihydroxy-N-(2-hydroxyethyl)Benzamide with 6-Aminopenicillanic Acid by Means of Biotransformation

Methods:

1.1 Preparation of the Enzyme Solution

Culturing of the Filamentous Fungi

The culturing of the filamentous fungi was first effected on slant agar (malt agar) at 30° C. for 7 d and then on malt agar plates at 30° C. for 7 d. Subsequently, three well colonized malt agar pieces each about 1 cm² in size were cut out of an agar plate culture with a sterile spatula and transferred into 300 ml Erlenmeyer flasks with 60 ml of BSM. This preculture was incubated at 30° C. as a standing culture for 7 days.

The mycelium formed in the glucose precultures of the filamentous fungi was homogenized three times for 10 s each by means of an Ultraturrax device at 17,000 rpm. For the experiments, 2.5 ml of the homogenizate was passaged into 100 ml Erlenmeyer flasks with 25 ml of BSM. The flasks and the medium were sterilized separately to avoid losses of medium during heat sterilization in the autoclave. Prior to inoculation, the medium was filled into the flasks. These charges were supplemented with a 0.050% sterile-filtered Tween 80 solution. The flasks were cultured in a shaking incubator at 158 rpm at 30° C.

Anion-Exchanger Chromatography

For enhanced secretion of the ligninolytical enzymes into cultures of T. versicolor and Pycnoporus cinnabarinus, veratryl alcohol was employed. Since this compound and its metabolites would interfere with later incubation experiments using culture filtrate, their were separated off using Q-Sepharose. Thus, 1 ml of the anion-exchange matrix was equilibrated three times with 100 ml of histidine buffer. Subsequently, the equilibrated Q-Sepharose was stirred with 80 ml of culture filtrate for 1 h slowly on a magnetic stirrer at room temperature. Thereafter, the aqueous supernatant was filtered off from the Q-Sepharose through a GF-6 glass fiber filter, the matrix was washed 20 times with 10 ml of histidine buffer each, and the protein was eluted with 15 ml of high-salt histidine buffer.

Ultrafiltration

For the experiments with culture filtrate to be comparable, the same enzyme activities should be employed in the incubation charges. Thus, it was necessary to concentrate the purified culture supernatants and to dilute them accordingly for the reactions. Concentration was effected by ultrafiltration using Centricon-30 and Centriprep-30 microconcentrators in a cooling centrifuge at 3000 rpm at 4° C. for 60 min.

Desalting

For the reactions with culture filtrate, it was necessary to desalt the purified and concentrated protein sample. Onto a packed Sephadex G-25 Superfine column, 2.5 ml of sample was charged and eluted with 3.7 ml of histidine buffer.

The protein eluate obtained was stored at −20° C.

1.2 Coupling Reaction

The 2,5-dihydroxy-N-(2-hydroxyethyl)benzamide is reacted at an equimolar ratio with 6-aminopenicillanic acid (1 mM) in sodium acetate buffer (pH 5; 0.02 M) under the influence of a laccase (975 nmol/2 ml/min; obtained from Trametes versicolor) for 35 min at room temperature. For promoting the conversion rate, the reaction solution is shaken at 100 rpm. For processing, the reaction solution is extracted using an octadecane solid phase. Elution of the product is effected with ethyl acetate or acetonitrile. The residue remaining after removal of the solvent is purified by high-performance liquid chromatography.

Result: Coupling of the antibiotic parent substance 6-aminopenicillanic acid with 2,5-dihydroxy-N-(2-hydroxyethyl)benzamide results in an antimicrobially active compound. The yield of coupling product is 66%.

Analysis of the compound obtained by means of mass-spectrometric methods yielded a mass of 411.

From the sum of the results of structural analysis, the following structure is to be assumed for the coupling product obtained:

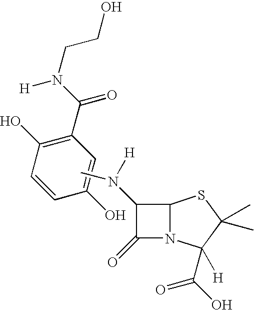

Figure: Proposed structure of the cross-coupling product

### EXAMPLE 2

Preparation of Novel Antimicrobially Active Substances by Coupling 2,5-dihydroxy-N-(2-hydroxyethyl)Benzamide with 7-Aminocephalosporinic Acid by Means of Biotransformation

Methods: The preparation of the enzyme solution was effected according to Example 1.

The 2,5-dihydroxy-N-(2-hydroxyethyl)benzamide is reacted at an equimolar ratio with 7-aminocephalosporinic acid (1 mM) in sodium acetate buffer (pH 5; 0.02 M) under the influence of a laccase (975 nmol/2 ml/min; obtained from Pycnoporus cinnabarinus) for 35 min at room temperature. For promoting the conversion rate, the reaction solution is shaken at 100 rpm. For processing, the reaction solution is extracted using an octadecane solid phase. Elution of the product is effected with ethyl acetate or acetonitrile. The residue remaining after removal of the solvent is purified by high-performance liquid chromatography.

Result: Coupling of the antibiotic parent substance 7-aminocephalosporinic acid with 2,5-dihydroxy-N-(2-hydroxyethyl)benzamide results in an as yet unknown antimicrobially active compound. The yield of coupling product is 68%.

### EXAMPLE 3

Preparation of Novel Antimicrobially Active Substances by Coupling 2,5-dihydroxy-N-(2-hydroxyethyl)Benzamide with 7-aminodeacetoxycephalosporinic Acid by Means of Biotransformation

Methods: The preparation of the enzyme solution was effected according to Example 1.

The 2,5-dihydroxy-N-(2-hydroxyethyl)benzamide is reacted at an equimolar ratio with 7-aminodeacetoxycephalosporinic acid (1 mM) in sodium acetate buffer (pH 5; 0.02 M) under the influence of a laccase (975 nmol/2 ml/min; obtained from Trametes versicolor) for 40 min at room temperature. For promoting the conversion rate, the reaction solution is shaken at 100 rpm. For processing, the reaction solution is extracted using an octadecane solid phase. Elution of the product is effected with ethyl acetate. The residue remaining after removal of the solvent is purified by high-performance liquid chromatography.

Result: Coupling of the antibiotic parent substance 7-aminodeacetoxycephalosporinic acid with 2,5-dihydroxy-N-(2-hydroxyethyl)benzamide results in an as yet unknown antimicrobially active compound. The yield of coupling product is 73%.

Analysis of the compound obtained by means of mass-spectrometric methods yielded a mass of 409. The results of the nuclear resonance spectroscopic analyses are summarized in the following Table. For comparing the ¹H NMR signals, 2,5-dihydroxy-N-(2-hydroxyethyl)benzamide was also examined. The measurements were effected in the solvent DMSO-d₆ with the internal standard TMS using an ARX 300 (Bruker, Karlsruhe, Germany).

TABLE
Comparison of the ¹H NMR spectra of the cross-coupling product 3 and
the starting compound 2,5-dihydroxy-N-(2-hydroxyethyl)benzamide

| | | Signals | | |
|---|---|---|---|---|
| | | [ppm] | | |
| Signals | | from 2,5-di- | | |
| [ppm] | | hydroxy-N- | | Number and |
| from cross- | Multiplicity, | (2-hydroxy- | Multiplicity, | type of |
| coupling | coupling | ethyl) | coupling | atoms, |
| product 3 | constant | benzamide | constant | assignment |
| 12.79 | s | — | — | 1H, COOH |
| 9.98 | s | 11.60 | s | 1H, OH at C2, |
| | | | | C₆H₂₍₃₎ |
| 9.66 | s | 8.98 | s | 1H, OH at C5, |
| | | | | C₆H₂₍₃₎ |
| 8.50 | t, ³J = 5.3 | 8.69 | t, ³J = 5.3 | 1H, NH, |
| | | | | CH₂—NH |
| 6.73 | s | 7.27 | d(s), | 1H, H at C6, |
| | | | ⁴ = 2.8 | C₆H₂₍₃₎ |
| 6.65 | s | 6.85 | dd, ³J = 8.7, | 1H, H at C4, |
| | | | ⁴J = 2.8 | C₆H₂₍₃₎ |
| — | | 6.73 | d, ³J = 8.7 | 1H, H at C3, |
| | | | | C₆H₂₍₃₎ |
| 6.42 | d, ³J = 9.3 | — | | 1H, NH at |
| | | | | β-lactam ring |
| 5.85 | t, ³J = 5.4 | 4.78 | t, ³J = 5.5 | 1H, OH, |
| | | | | CH₂—OH |
| 5.48 | m, ³J = 9.3, | — | | 1H, H7, |
| | ³J = 4.4 | | | β-lactam ring |
| 5.02 | m, ³J = 4.4 | — | | 1H, H6, |
| | | | | β-lactam ring |
| 3.63 | m | 3.54 | m | 2H, CH₂, |
| | | | | CH₂—OH |
| 3.49 | m | 3.36 | m | 2H, CH₂, |
| | | | | CH₂—NH |
| 3.29/3.50 | ABq, | — | | 2H, CH₂, |
| | ²J = 17.2 | | | heterocyclic |
| | | | | ring |
| 1.91 | s | — | | 3H, CH₃ |

From the sum of the results of structural analysis, the following structure is to be assumed for the coupling product obtained:

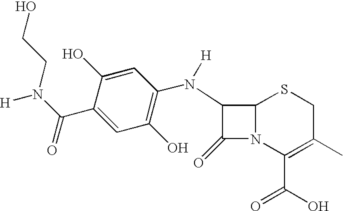

Figure: Proposed structure of the cross-coupling product

### EXAMPLE 4

Preparation of Novel Antimicrobially Active Substances by Coupling 2,5-dihydroxy-N-(2-hydroxyethyl)Benzamide with Amoxicillin by Means of Biotransformation

Methods: The preparation of the enzyme solution was effected according to Example 1.

The 2,5-dihydroxy-N-(2-hydroxyethyl)benzamide is reacted at an equimolar ratio with amoxicillin (1 mM) in sodium acetate buffer (pH 5; 0.02 M) under the influence of a laccase (975 nmol/2 ml/min; obtained from Trametes versicolor) for 50 min at room temperature. For promoting the conversion rate, the reaction solution is shaken at 100 rpm. For processing, the reaction solution is extracted using an octadecane solid phase. Elution of the product is effected with ethyl acetate or acetonitrile. The residue remaining after removal of the solvent is purified by high-performance liquid chromatography.

Result: Coupling of the antibiotic amoxicillin with 2,5-dihydroxy-N-(2-hydroxyethyl)benzamide results in an as yet unknown antimicrobially active compound. The yield of coupling product is 550/%.

Analysis of the compound obtained by means of mass-spectrometric methods yielded a mass of 560. The results of the nuclear resonance spectroscopic analyses are summarized in the following Table. For comparing the ¹H NMR signals, 2,5-dihydroxy-N-(2-hydroxyethyl)benzamide (see Table in Example 3) and amoxicillin were also examined. The measurements were effected in the solvent CD₃OD with the internal standard TMS using an ARX 300 (Bruker, Karlsruhe, Germany).

TABLE
Comparison of the ¹H NMR spectra of the cross-coupling product 4
and the starting compound amoxicillin

| Signals | | | | |
|---|---|---|---|---|
| [ppm] | | | | |
| from | | Signals | | Number |
| cross- | | [ppm] | | and type |
| coupling | | from | | of atoms, |
| product 4 | Multiplicity | amoxicillin | Multiplicity | assignment |
| 7.48 | d, ³J = 7.2 | 7.49 | d, ³J = 7.2 | 2H, h at C3/C5, |
| | | | | C₆H₄ |
| 6.85 | d, ³J = 7.2 | 6.83 | d, ³J = 7.2 | 2H, h at C2/C6, |
| | | | | C₆H₄ |
| 6.74 | d, ³J = 8.6 | — | — | 1H, H at C3, |
| | | | | C₆H₂ |
| 6.63 | d, ³J = 8.6 | — | — | 1H, H at C4, |
| | | | | C₆H₂ |
| 5.99 | m, ³J = 4.2 | 5.99 | m, ³J = 4.2 | 1H, H7 at |
| | | | | β-lactam ring |
| 5.92 | m, ³J = 4.2 | 5.92 | m, ³J = 4.2 | 1H, H6 at |
| | | | | β-lactam ring |
| 4.31 | s | 4.31 | s | 1H, CH, |
| | | | | heterocyclic ring |
| 3.70 | m | — | — | 2H, CH₂, |
| | | | | CH₂—OH |
| 3.49 | m | — | — | 2H, CH₂, |
| | | | | CH₂—NH |
| 1.57 | s | 1.57 | s | 3H, CH₃, |
| | | | | heterocyclic ring |
| 1.50 | s | 1.50 | s | 3H, CH₃, |
| | | | | heterocyclic ring |

From the sum of the results of structural analysis, the following structure is to be assumed for the coupling product obtained:

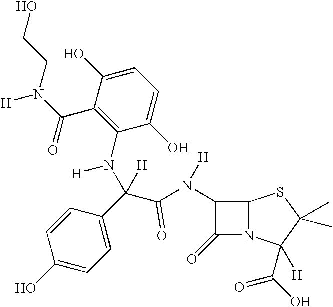

Figure: Proposed structure of the cross-coupling product

### EXAMPLE 5

Preparation of Novel Antimicrobially Active Substances by Coupling 2,5-dihydroxy-N-(2-hydroxyethyl)Benzamide with Ampicillin by Means of Biotransformation

Methods: The preparation of the enzyme solution was effected according to Example 1.

The 2,5-dihydroxy-N-(2-hydroxyethyl)benzamide is reacted at an equimolar ratio with ampicillin (1 mM) in sodium acetate buffer (pH 5; 0.02 M) under the influence of a laccase (975 nmol/2 ml/min; obtained from Trametes versicolor) for 50 min at room temperature. For promoting the conversion rate, the reaction solution is shaken at 100 rpm. For processing, the reaction solution is extracted using an octadecane solid phase. Elution of the product is effected with ethyl acetate or acetonitrile. The residue remaining after removal of the solvent is purified by high-performance liquid chromatography.

Result: Coupling of the antibiotic ampicillin with 2,5-dihydroxy-N-(2-hydroxyethyl)benzamide results in an as yet unknown antimicrobially active compound. The yield of coupling product is 75%.

Analysis of the compound obtained by means of mass-spectrometric methods yielded a mass of 544.

From the sum of the results of structural analysis, the following structure is to be assumed for the coupling product obtained:

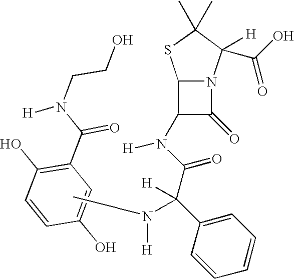

Figure: Proposed structure of the cross-coupling product

### EXAMPLE 6

Preparation of Novel Antimicrobially Active Substances by Coupling 2,5-dihydroxy-N-(2-hydroxyethyl)Benzamide with Sulfonamides by Means of Biotransformation

The 2,5-dihydroxy-N-(2-hydroxyethyl)benzamide is reacted at an equimolar ratio with p-aminobenzenesulfonamide (1 mM) in sodium acetate buffer (pH 5; 0.02 M) under the influence of a laccase (975 nmol/2 ml/min; obtained from Pycnoporus cinnabarinus) for 30 min at room temperature. For promoting the conversion rate, the reaction solution is shaken at 100 rpm. For processing, the reaction solution is extracted using an octadecane solid phase. Elution of the product is effected with ethyl acetate or acetonitrile. The residue remaining after removal of the solvent is purified by high-performance liquid chromatography.

Result: Coupling of the biologically active substance p-aminobenzenesulfonamide with 2,5-dihydroxy-N-(2-hydroxyethyl)benzamide results in an as yet unknown antimicrobially active compound. The yield of coupling product is 74%.

### EXAMPLE 7

Preparation of Novel Antimicrobially Active Substances by Coupling 2,5-dihydroxy-N-(2-hydroxyethyl)Benzamide with Substituted Salicylic Acids by Means of Biotransformation

The 2,5-dihydroxy-N-(2-hydroxyethyl)benzamide is reacted at an equimolar ratio with 4-amino-2-hydroxybenzoic acid (1 mM) in sodium acetate buffer (pH 5; 0.02 M) under the influence of a laccase (975 nmol/2 ml/min; obtained from, for example, Trametes versicolor or Pycnoporus cinnabarinus) for 50 min at room temperature. For promoting the conversion rate, the reaction solution is shaken at 100 rpm. For processing, the reaction solution is extracted using an octadecane solid phase. Elution of the product is effected with ethyl acetate or acetonitrile. The residue remaining after removal of the solvent is purified by high-performance liquid chromatography.

Result: Coupling of 4-amino-2-hydroxybenzoic acid with 2,5-dihydroxy-N-(2-hydroxyethyl)benzamide results in an as yet unknown antimicrobially active compound. The yield of coupling product is 73%.

### EXAMPLE 8

Preparation of Novel Antimicrobially Active Substances by Coupling 2,5-dihydroxy-benzoic Acids and Esters with 6-aminopenicillanic Acid by means of Biotransformation

Methods: The preparation of the enzyme solution was effected according to Example 1.

The 2,5-dihydroxybenzoic acid methyl ester is reacted at an equimolar ratio with 6-aminopenicillanic acid (1 mM) in sodium acetate buffer (pH 5; 0.02 M) under the influence of a laccase (975 nmol/2 ml/min; obtained from Pycnoporus cinnabarinus) for 1 h at room temperature. For promoting the conversion rate, the reaction solution is shaken at 100 rpm. For processing, the reaction solution is extracted using an octadecane solid phase. Elution of the product is effected with ethyl acetate or acetonitrile. The residue remaining after removal of the solvent is purified by high-performance liquid chromatography.

Result: Coupling of the antibiotic parent substance 6-aminopenicillanic acid with 2,5-dihydroxybenzoic acid methyl ester results in an as yet unknown antimicrobially active compound having a high activity. The yield of coupling product is 660/%.

### EXAMPLE 9

Preparation of Novel Antimicrobially Active Substances by Coupling 2,5-dihydroxy-benzoic Acids and Esters with 7-aminocephalosporinic Acid by Means of Biotransformation

Methods: The preparation of the enzyme solution was effected according to Example 1.

The 2,5-dihydroxybenzoic acid methyl ester is reacted at an equimolar ratio with 7-aminocephalosporinic acid (1 mM) in sodium acetate buffer (pH 5; 0.02 M) under the influence of a laccase (975 nmol/2 ml/min; obtained from, for example, Trametes versicolor) for 1 h at room temperature. For promoting the conversion rate, the reaction solution is shaken at 100 rpm. For processing, the reaction solution is extracted using an octadecane solid phase. Elution of the product is effected with ethyl acetate or acetonitrile. The residue remaining after removal of the solvent is purified by high-performance liquid chromatography.

Result: Coupling of the antibiotic parent substance 7-aminocephalosporinic acid with 2,5-dihydroxybenzoic acid methyl ester results in an as yet unknown antimicrobially active compound. The yield of coupling product is 63%.

### EXAMPLE 10

Preparation of Novel Antimicrobially Active Substances by Coupling 2,5-dihydroxy-benzoic Acids and Esters with 7-Aminodeacetoxycephalosporinic Acid by Means of Biotransformation

Methods: The preparation of the enzyme solution was effected according to Example 1.

The 2,5-dihydroxybenzoic acid methyl ester is reacted at an equimolar ratio with 7-aminodeacetoxycephalosporinic acid (1 mM) in sodium acetate buffer (pH 5; 0.02 M) under the influence of a laccase (975 nmol/2 ml/min; obtained from Pycnoporus cinnabarinus) for 1 h at room temperature. For promoting the conversion rate, the reaction solution is shaken at 100 rpm. For processing, the reaction solution is extracted using an octadecane solid phase. Elution of the product is effected with ethyl acetate or acetonitrile. The residue remaining after removal of the solvent is purified by high-performance liquid chromatography.

Result: Coupling of 2,5-dihydroxybenzoic acids and esters with 7-aminodeacetoxycephalosporinic acid results in an as yet unknown antimicrobially active compound. The yield of coupling product is 71%.

### EXAMPLE 11

Preparation of Novel Antimicrobially Active Substances by Coupling 2,5-dihydroxy-benzoic Acids and Esters with Amoxicillin by Means of Biotransformation

Methods: The preparation of the enzyme solution was effected according to Example 1.

The 2,5-dihydroxybenzoic acid methyl ester is reacted at an equimolar ratio with amoxicillin (1 mM) in sodium acetate buffer (pH 5; 0.02 M) under the influence of a laccase (975 nmol/2 ml/min; obtained from Trametes versicolor) for 45 min at room temperature. For promoting the conversion rate, the reaction solution is shaken at 100 rpm. For processing, the reaction solution is extracted using an octadecane solid phase. Elution of the product is effected with ethyl acetate or acetonitrile. The residue remaining after removal of the solvent is purified by high-performance liquid chromatography.

Result: Coupling of the antibiotic amoxicillin with 2,5-dihydroxybenzoic acid methyl ester results in an as yet unknown antimicrobially active compound. The yield of coupling product is 66%.

From the sum of the results of structural analysis, the following structure is to be assumed for the coupling product obtained:

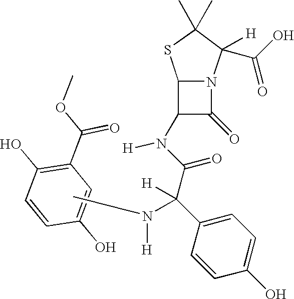

Figure: Proposed structure of the cross-coupling product

### EXAMPLE 12

Preparation of Novel Antimicrobially Active Substances by Coupling 2,5-dihydroxy-benzoic Acids and Esters with Amoxicillin by Means of Biotransformation

Methods: The preparation of the enzyme solution was effected according to Example 1.

The 2,5-dihydroxybenzoic acid methyl ester is reacted at an equimolar ratio with ampicillin (1 mM) in sodium acetate buffer (pH 5; 0.02 M) under the influence of a laccase (975 nmol/2 ml/min; obtained from Trametes versicolor) for 45 min at room temperature. For promoting the conversion rate, the reaction solution is shaken at 100 rpm. For processing, the reaction solution is extracted using an octadecane solid phase. Elution of the product is effected with ethyl acetate or acetonitrile. The residue remaining after removal of the solvent is purified by high-performance liquid chromatography.

Result: Coupling of the antibiotic ampicillin with 2,5-dihydroxybenzoic acid methyl ester results in an as yet unknown antimicrobially active compound. The yield of coupling product is 56%.

### EXAMPLE 13

Preparation of Novel Antimicrobially Active Substances by Coupling 2,5-dihydroxy-benzoic Acids and Esters with Sulfonamides by Means of Biotransformation

Methods: The preparation of the enzyme solution was effected according to Example 1.

The 2,5-dihydroxybenzoic acid methyl ester is reacted at an equimolar ratio with p-aminobenzenesulfonamide (1 mM) in sodium acetate buffer (pH 5; 0.02 M) under the influence of a laccase (975 nmol/2 ml/min; obtained, for example, from Trametes versicolor or Pycnoporus cinnabarinus) for 45 min at room temperature. For promoting the conversion rate, the reaction solution is shaken at 100 rpm. For processing, the reaction solution is extracted using an octadecane solid phase. Elution of the product is effected with ethyl acetate or acetonitrile. The residue remaining after removal of the solvent is purified by high-performance liquid chromatography.

Result: Coupling of the antibiotic p-aminobenzenesulfonamide with 2,5-dihydroxy-benzoic acid methyl ester results in an as yet unknown antimicrobially active compound. The yield of coupling product is 66%.

### EXAMPLE 14

Preparation of Novel Antimicrobially Active Substances by Coupling 2,5-dihydroxy-benzoic Acids and Esters with Substituted Salicylic Acids by Means of Biotransformation

Methods: The preparation of the enzyme solution was effected according to Example 1.

The 2,5-dihydroxybenzoic acid methyl ester is reacted at an equimolar ratio with 4-amino-2-hydroxybenzoic acid (1 mM) in sodium acetate buffer (pH 5; 0.02 M) under the influence of a laccase (975 nmol/2 ml/min; obtained, for example, from Trametes versicolor or Pycnoporus cinnabarinus) for 45 min at room temperature. For promoting the conversion rate, the reaction solution is shaken at 100 rpm. For processing, the reaction solution is extracted using an octadecane solid phase. Elution of the product is effected with ethyl acetate or acetonitrile. The residue remaining after removal of the solvent is purified by high-performance liquid chromatography.

Result: Coupling of 4-amino-2-hydroxybenzoic acid with 2,5-dihydroxybenzoic acid methyl ester results in an as yet unknown antimicrobially active compound. The yield of coupling product is 63%.

### EXAMPLE 15

Preparation of Novel Antimicrobially Active Substances by Coupling Hydrocaffeic Acid with 6-aminopenicillanic Acid by Means of Biotransformation

Methods: The preparation of the enzyme solution was effected according to Example 1.

3-(3,4-Dihydroxyphenyl)propionic acid is reacted at an equimolar ratio with 6-aminopenicillanic acid (1 mM) in sodium acetate buffer (pH 5; 0.02 M) under the influence of a laccase (975 nmol/2 ml/min; obtained from Pycnoporus cinnabarinus) for 2 h at room temperature. For promoting the conversion rate, the reaction solution is shaken at 100 rpm. For processing, the reaction solution is extracted using an octadecane solid phase. Elution of the product is effected with ethyl acetate or acetonitrile.

Result: Coupling of the antibiotic parent substance 6-aminopenicillanic acid with hydrocaffeic acid results in an as yet unknown antimicrobially active compound having a high activity.

### EXAMPLE 16

Preparation of Novel Antimicrobially Active Substances by Coupling 2,5-dihydroxy-N-(2-hydroxyethyl)Benzamide with Mercaptanes by Means of Biotransformation

Methods: The preparation of the enzyme solution was effected according to Example 1.

The 2,5-dihydroxy-N-(2-hydroxyethyl)benzamide is reacted at an equimolar ratio with 3-mercapto-1,2,4-triazole (1 mM) in sodium acetate buffer (pH 5; 0.02 M) under the influence of a laccase (975 nmol/2 ml/min; obtained, for example, from Trametes versicolor or Pycnoporus cinnabarinus) for 50 min at room temperature. For promoting the conversion rate, the reaction solution is shaken at 100 rpm. For processing, the reaction solution is extracted using an octadecane solid phase. Elution of the product is effected with ethyl acetate or acetonitrile. The residue remaining after removal of the solvent is purified by high-performance liquid chromatography.

Result: Coupling of 3-mercapto-1,2,4-triazole with 2,5-dihydroxy-N-(2-hydroxyethyl)benzamide results in an as yet unknown antimicrobially active compound. The yield of coupling product is 45%.

### EXAMPLE 17

Preparation of Novel Antimicrobially Active Substances by Coupling 2,5-dihydroxy-N-(2-hydroxyethyl)Benzamide with Mercaptanes by Means of Biotransformation

Methods: The preparation of the enzyme solution was effected according to Example 1.

The 2,5-dihydroxy-N-(2-hydroxyethyl)benzamide is reacted at an equimolar ratio with 2-mercaptonicotinic acid (1 mM) in sodium acetate buffer (pH 5; 0.02 M) under the influence of a laccase (975 nmol/2 ml/min; obtained, for example, from Trametes versicolor or Pycnoporus cinnabarinus) for 50 min at room temperature. For promoting the conversion rate, the reaction solution is shaken at 100 rpm. For processing, the reaction solution is extracted using an octadecane solid phase. Elution of the product is effected with ethyl acetate or acetonitrile. The residue remaining after removal of the solvent is purified by high-performance liquid chromatography.

Result: Coupling of 2-mercaptonicotinic acid with 2,5-dihydroxy-N-(2-hydroxyethyl)benzamide results in as yet unknown antimicrobially active compounds. The yield of coupling products is 55%.

### EXAMPLE 18

Examination of the Coupling Products According to the Invention for Antimicrobial Activity

Methods: An agar diffusion test according to Burkhardt was performed (Burkhardt, F. (Ed.): Mikrobiologische Diagnostik, Georg-Thieme-Verlag Stuttgart, N.Y., 1992, p. 724). The test substances are applied to Sensi-Disc test plates (Becton Dickinson Microbiology Systems, Cockeysville, USA) in graduated concentrations. For testing, Mueller-Hinton II agar was employed in Stacker Petri dishes (Becton Dickinson Microbiology Systems, Cockeysville, USA). S. aureus strain ATCC 6538 was selected as a test strain. The seeding rate of this test strain was selected in such a way that densely arranged, nut non-confluent individual colonies developed after from 16 to 20 h of incubation. After drying the inoculated nutrient substrates, a maximum of 6 test plates is placed onto the agar surface with slight pressing. After 18+2 h of incubation at 36° C., the inhibition halos are measured. For internal quality control, the method was checked with S. aureus ATCC 25923.

Results: The active substances obtainable by the biotransformation according to the invention are antimicrobially active (Table 1). The highest antimicrobial activity is observed in the coupling products according to Examples 4, 9 and 10.

TABLE 1
Antibacterial activity in an agar diffusion test according to Burkhardt
against S. aureus ATCC 6538

| | Inhibition halo (mm) | | | |
|---|---|---|---|---|
| Active Substance | 250 μg | 125 μg | 50 μg | 10 μg |
| Coupling product of 6-aminopeni- | 24 | 22 | 18 | 12 |
| cillanic acid and 2,5-dihydroxy-N- | | | | |
| (2-hydroxyethyl)benzamide | | | | |
| according to Example 1 | | | | |
| Coupling product of | 22 | 18 | 14 | r |
| 7-aminocephalosporinic acid and 2,5- | | | | |
| dihydroxy-N-(2-hydroxyethyl)benz- | | | | |
| amide according to Example 2 | | | | |
| Coupling product of 7- | 24 | 22 | 18 | r |
| aminodeacetoxycephalosporinic acid | | | | |
| and 2,5-dihydroxy-N-(2-hydroxy-ethyl) | | | | |
| benzamide according to Example 3 | | | | |
| Coupling product of amoxicillin and | >30 | >30 | >30 | >30 |
| 2,5-dihydroxy-N-(2-hydroxyethyl) | | | | |
| benzamide according to Example 4 | | | | |
| Coupling product of | 22 | 20 | r | r |
| p-aminobenzenesulfonamide and | | | | |
| 2,5-dihydroxy-N-(2-hydroxyethyl)benz- | | | | |
| amide according to Example 6 | | | | |
| Coupling product of | 10 | r | r | r |
| 4-amino-2-hydroxybenzoic acid and | | | | |
| 2,5-dihydroxy-N-(2-hydroxyethyl)- | | | | |
| benzamide according to Example 7 | | | | |
| Coupling product of 6-aminopenicil- | | 30 | 24 | r |
| lanic acid and 2,5-dihydroxybenzoic | | | | |
| acid methyl ester according to | | | | |
| Example 8 | | | | |
| Coupling product of | | 10 | r | r |
| 7-aminocephalosporinic acid and | | | | |
| 2,5-dihydroxybenzoic acid methyl | | | | |
| ester according to Example 9 | | | | |
| Coupling product of | | 30 | 14 | r |
| 2,5-dihydroxybenzoic acid methyl | | | | |
| ester and 7-aminodeacetoxycephalo- | | | | |
| sporinic acid according to Example 10 | | | | |
| Coupling product of amoxicillin and | >30 | >30 | >30 | >30 |
| 2,5-dihydroxybenzoic acid methyl | | | | |
| ester according to Example 11 | | | | |
| Coupling product of | 10 | r | | |
| p-aminobenzenesulfonamide and | | | | |
| 2,5-dihydroxybenzoic acid methyl | | | | |
| ester according to Example 13 | | | | |
| Coupling product of | 10 | r | | |
| 4-amino-2-hydroxybenzoic acid | | | | |
| and 2,5-dihydroxybenzoic acid methyl | | | | |
| ester according to Example 14 | | | | |

### EXAMPLE 19

Examination for Antimicrobial Activity Against Multiresistant Germs

Methods: The examination was effected by an agar diffusion test according to the method described in Example 13. As a test germ, the multiresistant S. aureus reference strain “Norddeutscher Stamm” (Smith Kline Beecham) was employed.

Results: Surprisingly, the coupling products are also, in part highly, effective against multiresistant germs (Table 2). The activity of the starting substances is clearly exceeded (Table 4).

TABLE 2
Antibacterial activity in an agar diffusion test according to Burkhardt
against S. aureus “Norddeutscher Stamm”

| | Inhibition halo (mm) | | | |
|---|---|---|---|---|
| Active Substance | 250 μg | 125 μg | 50 μg | 10 μg |
| Coupling product of | 20 | 18 | 16 | r |
| 6-aminopenicillanic acid and | | | | |
| 2,5-dihydroxy-N-(2-hydroxyethyl) | | | | |
| benzamide according to Example 1 | | | | |
| Coupling product of | 28 | 24 | 20 | r |
| 7-aminocephalosporinic acid and 2,5- | | | | |
| dihydroxy-N-(2-hydroxyethyl)benz- | | | | |
| amide according to Example 2 | | | | |
| Coupling product of 7-amino- | 24 | 22 | 20 | r |
| deacetoxycephalosporinic acid and | | | | |
| 2,5-dihydroxy-N-(2-hydroxyethyl) | | | | |
| benzamide according to Example 3 | | | | |
| Coupling product of amoxicillin and | 24 | 22 | 18 | 10 |
| 2,5-dihydroxy-N-(2-hydroxyethyl) | | | | |
| benzamide according to Example 4 | | | | |
| Coupling product of | 26 | 22 | 18 | r |
| p-aminobenzenesulfonamide and 2,5- | | | | |
| dihydroxy-N-(2-hydroxyethyl)benz- | | | | |
| amide according to Example 6 | | | | |
| Coupling product of 4- | r | r | r | r |
| amino-2-hydroxybenzoic acid and | | | | |
| 2,5-dihydroxy-N-(2-hydroxyethyl)- | | | | |
| benzamide according to Example 7 | | | | |
| Coupling product of 6- | | 16 | r | r |
| aminopenicillanic acid and 2,5- | | | | |
| dihydroxybenzoic acid methyl ester | | | | |
| according to Example 8 | | | | |
| Coupling product of | | r | r | r |
| 7-aminocephalosporinic acid | | | | |
| 2,5-dihydroxybenzoic acid methyl | | | | |
| ester according to Example 9 | | | | |
| Coupling product of 2,5-di- | | r | r | r |
| hydroxybenzoic acid methyl ester and | | | | |
| 7-aminodeacetoxycephalosporinic | | | | |
| acid according to Example 10 | | | | |
| Coupling product of amoxicillin | 20 | 16 | 10 | R |
| and 2,5-dihydroxybenzoic acid | | | | |
| methyl ester according to | | | | |
| Example 11 | | | | |
| Coupling product of | 16 | 12 | | |
| p-aminobenzenesulfonamide and | | | | |
| 2,5-dihydroxybenzoic acid methyl | | | | |
| ester according to Example 13 | | | | |
| Coupling product of | r | r | | |
| 4-amino-2-hydroxybenzoic acid | | | | |
| and 2,5-dihydroxybenzoic acid | | | | |
| methyl ester according to Example 14 | | | | |

### EXAMPLE 20

Methods: The examination was effected by an agar diffusion test according to the method described in Example 13. As a test germ, the multiresistant coagulase-negative S. epidermidis strain 99847 isolated from patient material was employed.

Results: The coupling products according to the invention are also effective against the difficult-to-control coagulase-negative staphylococci which play a role, for example, in the colonialization of catheter materials and therefore belong to the nosocomial problem germs. The activity of the starting substances is clearly exceeded (Table 4).

TABLE 3
Antibacterial activity in an agar diffusion test according to Burkhardt
against S. epidermidis

| | Inhibition halo (mm) | | | |
|---|---|---|---|---|
| | 250 | 125 | 50 | 10 |
| Active Substance | μg | μg | μg | μg |
| Coupling product of 6-aminopenicillanic acid | r | r | r | r |
| and 2,5-dihydroxy-N-(2-hydroxyethyl)benzamide | | | | |
| according to Example 1 | | | | |
| Coupling product of 7-aminocephalosporinic | 24 | 22 | r | r |
| acid and 2,5-dihydroxy-N-(2-hydroxyethyl)benz- | | | | |
| amide according to Example 2 | | | | |
| Coupling product of 7-aminodeacetoxycephalo- | 20 | 24 | r | r |
| sporinic acid and 2,5-dihydroxy-N-(2-hydroxy- | | | | |
| ethyl)benzamide according to Example 3 | | | | |
| Coupling product of amoxicillin and 2,5- | 30 | 28 | 18 | r |
| dihydroxy-N-(2-hydroxyethyl)benzamide | | | | |
| according to Example 4 | | | | |
| Coupling product of p-aminobenzenesulfonamide | 26 | 20 | r | r |
| and 2,5-dihydroxy-N-(2-hydroxyethyl)benz | | | | |
| amide according to Example 6 | | | | |
| Coupling product of 4-amino-2-hydroxybenzoic | r | r | r | r |
| acid and 2,5-dihydroxy-N-(2-hydroxyethyl)- | | | | |
| benzamide according to Example 7 | | | | |
| Coupling product of 6-aminopenicillanic acid and | | | | |
| 2,5-dihydroxybenzoic acid methyl ester accord- | | | | |
| ing to Example 8 | | | | |
| Coupling product of 7-aminocephalosporinic acid | | 14 | r | r |
| and 2,5-dihydroxybenzoic acid methyl ester | | | | |
| according to Example 9 | | | | |
| Coupling product of 2,5-dihydroxybenzoic acid | | r | r | r |
| methyl ester and 7-aminodeacetoxycephalo- | | | | |
| sporinic acid according to Example 10 | | | | |
| Coupling product of amoxicillin and 2,5- | 20 | 20 | 14 | r |
| dihydroxybenzoic acid methyl ester according to | | | | |
| Example 11 | | | | |
| Coupling product of p-aminobenzenesulfonamide | 12 | r | | |
| and 2,5-dihydroxybenzoic acid methyl ester | | | | |
| according to Example 13 | | | | |
| Coupling product of 4-amino-2-hydroxybenzoic | r | r | | |
| acid and 2,5-dihydroxybenzoic acid methyl ester | | | | |
| according to Example 14 | | | | |

TABLE 4
Antimicrobial activity of the starting materials

| | | Size of inhibition halo (mm) | |
|---|---|---|---|
| | Concentration/ | S. aureus. | |
| Active substance | disc | “Nord. Stamm” | S. epidermidis |
| Ethyl acetate | 0.25 | r | r |
| 2,5-Dihydroxybenzoic | 0.25 mg | r | r |
| acid methyl ester | | | |
| 2,5-Dihydroxy-N-(2- | 0.25 mg | 22 | 22 |
| hydroxyethyl) | 0.05 mg | 14 | r |
| benzamide | | | |
| p-Aminobenzene- | 0.25 mg | r | r |
| sulfonamide | | | |
| 4-Amino-2- | 0.25 mg | r | r |
| hydroxybenzoic | | | |
| acid | | | |
| Amoxicillin | 0.25 mg | 16 | 24 |
| | 0.12 mg | 14 | 20 |
| | 0.05 mg | 10 | 18 |
| | 0.01 mg | r | 12 |

### EXAMPLE 21

Method for the Stabilization of the Coupling Products According to the Invention

For increasing the stability of the cross-coupling product of an equimolar charge of amoxicillin and 2,5-dihydroxybenzoic acid methyl ester, prepared according to Example 10, a special extraction method on octadecane solid phases has been developed. Thus, the solid phase was activated by means of methanol and equilibrated with distilled water. Subsequently, x ml of the aqueous cross-coupling charge was applied and, after binding of the components, dried in a stream of air for 20 min. This is followed by a washing step with a mixture of 10% methanol/90% distilled water. Elution of the cross-coupling product was effected with acetonitrile which was also 100% degassed. The thus obtained extract was stored in the dark at 4° C.

Result: The coupling product is stable over the observation period of 21 days.

1. A process for the preparation of biologically active compounds from substances which bear at least one amino group and/or are aromatic compounds having only one hydroxy group or a heteroaromatic compound or which have in the molecule at least one aromatic ring with at least two hydroxy groups and at least one further substituent as well as corresponding quinoid compounds, characterized in that heteromolecularly linked coupling products having additional functional groups and/or a modified spectrum of activity and/or modified application properties become obtainable by a one-electron reaction catalyzed by free-radical forming enzymes of the classification according to the International Enzyme Nomenclature (Enzyme Nomenclature, Academic Press, Inc., 1992, p. 24-154) of EC 1.10.3.2 (laccase), EC 1.16.3.1 (ceruloplasmin), EC 1.11.1.13 (manganese peroxidases), EC 1.11.17 (peroxidases), EC 1.14.99.1 (monophenol-monooxygenase), EC 1.10.3.3 (ascorbate oxidase) and/or mixtures of these enzymes and/or supernatants of ligninolytic fungi and other microbial cultures having enzymatic activity, wherein the following can be introduced as additional functional groups:
A) synthones which provide the active substance with an altered dissolving behavior in aqueous and lipophilic systems and/or with a high interfacial activity;
B) aromatic molecules, heteroaromatic compounds;
C) heterocyclic compounds;
D) active substances having an independent biological activity;
E) active substances combining an independent activity and surfactant properties in one molecule.

2. The process according to claim 1, wherein said substrates are β-lactam antibiotics, tetracyclin antibiotics, anthracyclin antibiotics, polyene antibiotics, aminoglycosides, benzofuran derivatives, sulfonamides, quinoid compounds and organic acids.

3. The process according to claim 1, wherein said aqueous solution has a pH value of from 2 to 8 at temperatures of between 5° C. and 60° C.

4. The process according to claim 1, wherein said polyfunctional synthones mentioned under A) are C₁-C₁₈ Alkyl, C₁-C₁₈ alkenyl, C₁-C₁₈ alkynyl, C₁-C₁₈ alkoxy, C₁-C₁₈ oxycarbonyl, C₁-C₁₈ oxoalkyl, C₁-C₁₈ alkylsulfanyl, C₁-C₁₈ alkylsulfonyl, C₁-C₁₈ alkylimino or alkylamino synthones.

5. The process according to claim 1, wherein said aromatic molecules mentioned under B) are preferably mono-, di- or polycyclic aromatic synthones, each optionally provided with one or more functional groups or substituents selected from the group of halogens; sulfo; sulfone; sulfamino; sulfanyl; amino; amido; nitro; azo; imino; carboxy; cyano; formyl; hydroxy; halocarbonyl; carbamyl; carbamidoyl; phosphone; phosphonyl; C₁₋₁₈ alkyl; C₁₋₁₈ alkenyl; C₁₋₁₈ alkinyl; C₁₋₁₈ alkoxy; C₁₋₁₈ oxycarbonyl; C₁₋₁₈ oxoalkyl; C₁₋₁₈ alkylsulfanyl; C₁₋₁₈ alkylsulfonyl; C₁₋₁₈ alkylimino or alkylamino substituents.

6. The process according to claim 5, wherein said aromatic molecules mentioned under B) are mono-, di- or polycyclic aromatic compounds are those selected from the group consisting of anthracene, azulene, benzene, benzofuran, benzothiazole, benzothiazoline, carboline, carbazole, cinnoline, chroman, chromene, chrysene, fulvene, furan, imidazole, indazole, indene, indole, indoline, indolizine, isothiazole, isoquinoline, isoxazole, naphthalene, naphthylene, naphthylpyridine, oxazole, perylene, phenanthrene, phenazine, phthalizine, pteridine, purine, pyran, pyrazole, pyrene, pyridazine, pyridazone, pyridine, pyrimidine, pyrrole, quinazoline, quinoline, quinoxaline, sulfonyl, thiophene and triazine, each of which may be substituted.

7. The process according to claim 1, wherein said heterocyclic compounds mentioned under C) are pyrrolidine, pyridine, quinolizidine, quinoline, isoquinoline, indole, acridine, quinazoline and purine, each optionally provided with one or more functional groups or substituents selected from the group of halogens, sulfo, sulfone, sulfamino, sulfanyl, amino, amido.

8. The process according to claim 1, wherein said active substances mentioned under D) are imidazoles, azoles, flavones, isoflavones, tetracyclines, amino acid analogues and nucleotide analogues.

9. The process according to at least one of claims 1 to 8, characterized in that a change of optimum pH is achieved by the use of recombinant laccases.

10. The process according to at least one of claims 1 to 9, characterized in that said coupling reaction is optionally performed in the presence of at least one mediator selected from the group of hydroxylamines and/or hydroxamic acids and optionally a mediator selected from the group of amides.

11. The process according to claims 1 to 10, characterized in that the reaction products are stabilized after completion of the reaction.

12. Compounds obtainable from active substances of different classes of substances by a biotransformation according to claims 1 to 11.

13. Use of biologically active compounds according to claim 12 in human and veterinary medicine and in plant protection.

14. Use of biologically active compounds according to claims 12 and/or 13 in human and veterinary medicine and in plant protection as a sole active ingredient or in the form of combination preparations.

15. Use of biologically active compounds according to claims 12 to 14 for controlling infectious diseases, preferably as an agent having antimicrobial and antiviral effects for topical and/or systemic application.

16. Use of biologically active compounds according to claims 12 to 14 in infectious diseases with multiresistant Gram-positive germs.

17. Use of biologically active compounds according to claims 12 to 14 in infectious diseases with multiresistant Gram-negative germs.

18. Use of biologically active compounds according to claims 12 to 14 as an antimycotic agent for topical and/or systemic application in human and veterinary medicine and in plant protection.

19. Use of biologically active compounds according to claims 12 to 14 as a cytostatic agent.

20. The use according to any of claims 12 to 14, characterized in that the compounds obtained upon biotransformation are employed alone or in combination with one another.

21. Medicaments containing one or more of the compounds according to any of claims 12 to 19.

22. Medicaments containing one or more of the compounds according to any of claims 12 to 16 for preparing a medicament for the treatment of infectious diseases with Gram-positive pathogens.

23. Plant protective agents containing one or more of the compounds according to any of claims 12 to 18.

24. Use of the compounds according to any of the preceding claims as a surface-active agent.
